(19) United States
(12) Patent Application Publication
(10) Pub. No.: US 2010/0082006 A1
(43) Pub. Date: Apr. 1, 2010

(54) DEVELOPMENTALLY DESIGNED DIAPER

(76) Inventors: Mary S. Rogone, Apple Valley, CA (US); Philip N. Rogone, Apple Valley, CA (US); Kenneth S. Croteau, Hesperia, CA (US); Winnifred D. Croteau, Hesperia, CA (US)

Correspondence Address: KLEIN, O'NEILL & SINGH, LLP, 43 CORPORATE PARK, SUITE 204, IRVINE, CA, 92606, US

(21) Appl. No.: 12/589,514

(22) Filed: Apr. 28, 2009

Related U.S. Application Data
(60) Provisional application No. 61/127,209, filed on May 9, 2008.
(60) Provisional application No. 61/127,210, filed on May 9, 2008.
(60) Provisional application No. 61/127,211, filed on May 9, 2008.

(51) Int. Cl. A61F 13/15

(52) U.S. Cl. 604/374; 604/378

(57) ABSTRACT

The present disclosure provides a developmentally designed diaper for LBW infants that is based on NIDCAP principals to allow for the proper positioning of a LBW infants limbs while wearing the diaper.

### CROSS-REFERENCE TO RELATED APPLICATIONS

This application claims the benefit and priority of U.S. Provisional Application Nos. 61/127,209, 61/127,210, 61/127,211, each tiled on May 9, 2008, which are herein incorporated by reference in their entirety for all purposes.

### BACKGROUND

1. Field of the Invention

This disclosure is related to diapers, and more specifically to developmentally designed diapers for low birth weight, very low birth weight and extremely low birth weight infants.

2. Related Art

Center positioning, assessment and overall neonatal stress reduction and activity grouping interaction is of great importance to the care of low birth weight (LBW), very low birth weight (VLBW) and extremely low birth weight (ELBW) infants (hereinafter, collectively, “LBW infants”) adding in total to a more rapid recuperative course for the LBW infants.

Because of the musculo-skeletal problems associated with poor positioning of LBW infants, center positioning of the infant's limbs as close to normal as possible is the preferred position for this weight group. Generally, diapers now in use are too large in the crotch area for LBW infants because of the volume of diaper material being used liar absorbency considerations and the like. Thus, these diapers tend to spread a LBW infant's lower limbs beyond what is consider by the Newborn Individualized Developmental Care and Assessment Program (NIDCAP) developmental guide to be developmentally proper for this weight group. Unfortunately, there are no known commercial diapers currently on the market that allow an LBW infant's lower limbs to be moved closer together than about 1⅜ inches. In perspective this dimension is quite large when considering the actual size of the LBW infant's hips.

In addition, almost all commercial diapers contain a type of powder gel material that interacts with fluid by binding with the fluid, This generally allows the diaper to retain relatively large amounts of fluid, causing the diaper to swell in all directions. The swelling of the diaper exacerbates the limb positioning problem. Moreover, the use of gels in existing commercial diapers precludes using the diaper as a means of obtaining urine specimens for bedside spot checks of laboratory measurements. This makes it necessary for health care professionals to turn to stressful and invasive types of procedures to obtain fluid samples from LBW infants.

For these reasons, what is needed is a diaper for infants that is designed to properly fit the LBW infant to avoid debilitating developmental problems and which allow for the collection of urine specimens and the like.

### SUMMARY

The present disclosure provides a developmentally designed diaper (hereinafter, the “diaper”) for LBW infants that is based on NIDCAP principals to allow for the proper positioning of a LBW infants limbs while wearing the diaper. Since the diaper serves as an adjunct tool to contribute to an environment that is NIDCAP strategy based, the diaper is an important contribution to an individualized developmental care environment of the LBW infant that contributes to better patient outcomes.

In one aspect, the disclosure provides a developmentally designed diaper for LBW infants. The diaper includes an outer layer having the dimensions L₂, D₁, D₂, and D₃; an absorbent pad having the dimensions L₁, P₁, P₂, P₃ and P₄; a wick away liner disposed on top of the absorbent pad; and an absorbent inner liner that pulls moisture away from an infants skin to which it makes contact past the wick away liner and into the absorbent pad. The dimensions provided below, are specific to each developmentally designed diaper so that the diaper may be constructed specifically for a weight range for the extremely low birth weight infant (ELBW) of <2 lb 3 oz, the very low birth weight infant (VLBW) of up to <3 lb 3 oz) and the low birth weight infant (LBW) of up to <4.5 lb.

The diaper may be constructed out of specific materials and designed to be used by the bedside caregiver as a developmental tool to assist in monitoring changes in lab values, providing bedside spot checks of body fluids and providing the caregiver with a tool that adheres to NIDCAP Developmental guided interventions of which, center positioning, assessment and overall neonatal stress reduction and activity grouping interaction is of great importance adding in total to a more rapid recuperative course for the LBW neonatal infant. The NIDCAP Developmental guide is herein incorporated by reference for all purposes.

Advantageously, no need for plastics, tapes or absorbent polymers that interact with the infants skin used in the diaper.

These and other features and advantages of the present invention will be more readily apparent from the detailed description of the embodiments set forth below taken in conjunction with the accompanying drawings.

### BRIEF DESCRIPTION OF THE DRAWINGS

The present invention may he better understood, and its numerous objects, features, and advantages made apparent to those skilled in the art by referencing the accompanying drawings.

FIG. 1 is a simplified perspective view of a developmentally designed diaper in accordance with an embodiment of the present invention;

FIG. 2 is a simplified plan view of the diaper of FIG. 1; and

FIG. 3 is a simplified plan view of an absorbent pad in accordance with an embodiment of the present invention;

The use of the same reference symbols in different drawings indicates similar or identical items.

### DETAILED DESCRIPTION

All measurements stated in this application are important due to the nature of the diaper, the measurements in this summary are nonetheless approximations and are not intended to be specifically limiting.

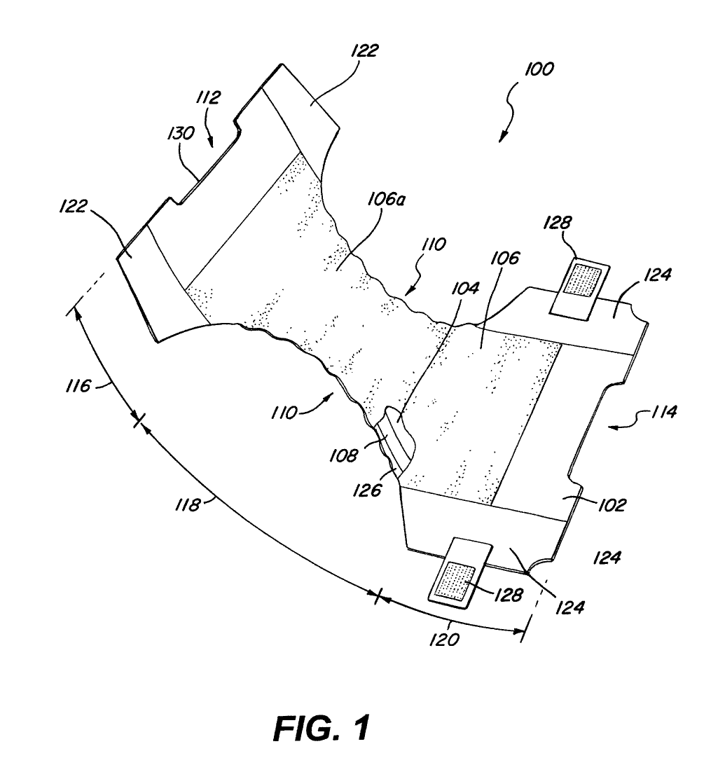

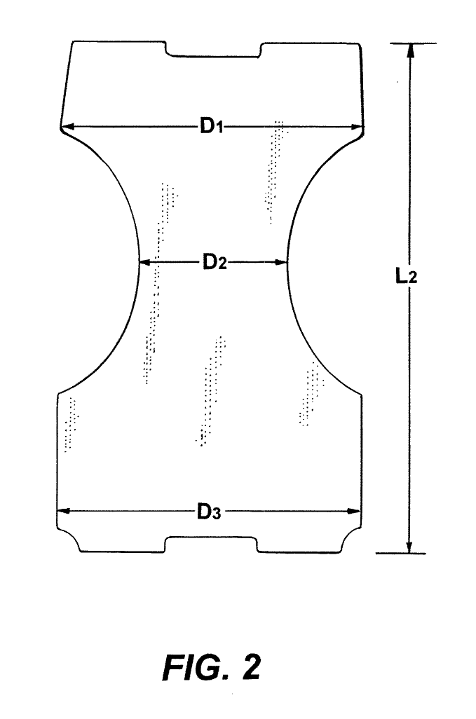

FIG. 1 is a perspective view showing a disposable diaper 100 under the contraction of elastic members (discussed below) n accordance with an embodiment of the invention. FIG. 2 is a simplified plan view showing the diaper 100, The diaper 100 includes an outer layer 102 and a body fluid absorbent pad 104 attached to an absorbent liner 106. The absorbent liner 106 may be made of a non woven water permeable layer, such as a polymer polyacrylate cross link, or a thin quickly absorbent, non woven polypropylene fiber liner or other similar material. Preferably, the absorbent liner 106 accounts for between approximately 14-16% of the diaper 100 to pull moisture rapidly away from the LBW infant's skin toward and into the absorbent pad 104.

A back-sheet 108 made of non-woven fabric bonded and may be bonded to an inner surface of the absorbent liner 106. The back-sheet 108 may be a water proof non-woven material, which accounts for between approximately 23-25% of the diaper 100.

As shown in FIG. 1, the diaper 100 is contoured by a pair of side edges 110 extending along the longitudinal direction and a pair of ends 112 and 114 extending: in the transverse direction. As for the configuration, the diaper 100 defines a front waist region 116 extending forward from a crotch region 118, and a rear waist region 120 extending rearward from the crotch region 118. A pair of front wings 122 extend in the transverse direction from the respective side edges 110 of the front waist region 116 and a pair of rear wings 124 extend in the transverse direction from the respective side edges 110 of the rear waist region 120. At end 112 is positioned an umbilical cord cutout 130, The cutout may range from between about 6.5 cm×2 cm for the ELBW infant sized diaper to about 7 cm×2 cm for the LBW infant sized diaper.

Elastic members 126 extending in the longitudinal direction are interposed and secured between the absorbent liner 106 and the backsheet 108 along the respective side edges 110. The front and rear wings 122, 124 may be made of a non-woven fabric, a plastic film or a laminate thereof. The rear wings 124 are provided on respective inner surfaces with fasteners 128 formed from hook and loop fasteners and the like. In one embodiment, the diaper 100 uses a low profile hook and loop system to secure the diaper to the infant along with the elastic members 126 to stem leakage of fluid. In one embodiment, the hook and loop system includes 5 cm (L)×2 cm (W) tabs 128 on each side of wings 124 connecting to an 8 cm×3 cm strip provided on the outer surface of wings 122, Advantageously, there is no need for plastics, tapes or absorbent polymers that interact with the infants skin used in the diaper.

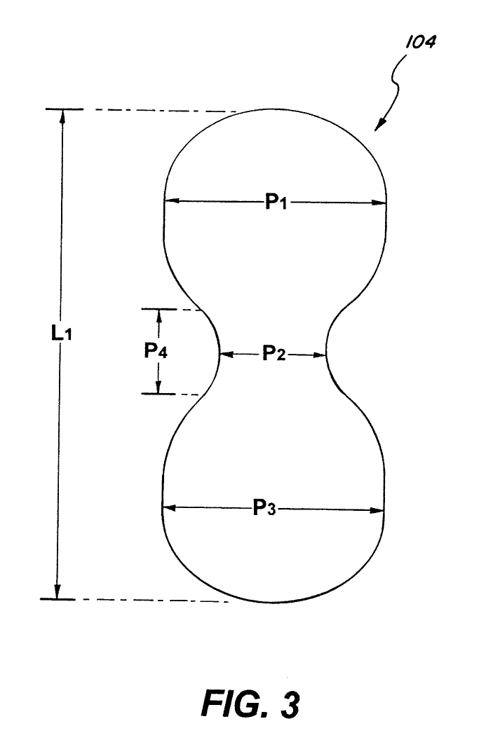

FIG. 3 is a simplified plan view of the body fluid absorbent pad 104 in accordance with an embodiment. The absorbent pad 104 includes a chlorine free sulphate cellulose pulp pad or the like. The absorbent pad 104 accounts for approximately 56% of the diaper material. In one embodiment, the absorbent pad 104 may also include a pass through wick away liner 106a disposed on top of the absorbent pad 104. The flick away liner may account for approximately 1% of the diaper material.

In one embodiment, a hot melt adhesive hydrocarbon oil or similar adhesive comprising approximately 0.4% of the diaper material is used to bond the various sheets and liners.

The following provides specific embodiments of the diaper 100 as constructed for each birth weight range. Referring now to FIGS. 2 and 3 certain dimension of the diaper 100 are important to the effectiveness of the developmentally designed diaper.

Diaper Specifications for the ELBW Infant

| | |
|---|---|
| Weight | 5 grams +/− 2.5 gm |
| Pad length (L₁) | 15 cm +/− 3.0 cm |
| Pad width (P₁, P₃) | 7 cm +/− 2 cm |
| Pad crotch size (P₂ × P₄) | 2.5 cm × 2 cm +/− 1.0 cm |
| Pad material | chlorine free sulphate cellulose pulp |
| Outer layer material | Non woven material |
| Non woven length (L₂) | 19 cm +/− 2.5 cm |
| Non woven width (D₁, D₃) | 15 cm +/− 2.5 cm |
| Non woven crotch width (D₂) | 10.5 cm +/− 3.0 cm |
| Elastic | Double elastic 10 cm +/− 2.0 cm |
| Inner liner | 11.5 cm × 6 cm +/− 2.5 cm |
| Absorption | 40 CC of NACL in testing +/− 10 cc |

The following provides specific embodiments of the diaper 100 as constructed for the Very Low Birth Weight Infant. Referring now to FIGS. 2 and 3 certain dimension of the diaper 100 are important to the effectiveness of the developmentally designed chapel.

Diaper Specifications VLBW Infant

| | |
|---|---|
| Weight | 8.5 grams +/− 2.5 gm |
| Pad length (L₁) | 20.5 cm +/− 3.0 cm |
| Pad width (P₁, P₃) | 9 cm +/− 2.0 cm |
| Pad crotch size (P₂ × P₄) | 4 cm × 3 cm +/− 1.5 cm |
| Pad material | chlorine free sulphate cellulose pulp |
| Outer layer material | Non woven material |
| Non woven length (L₂) | 24 cm +/− 2.5 in |
| Non woven width (D₁, D₃) | 19 cm +/− 2.5 cm |
| Non woven crotch width (D₂) | 12 cm +/− 3.0 cm |
| Elastic | Double elastic 16 cm +/− 2.0 cm |
| Inner liner | 13 cm × 6 cm +/− 2.5 cm |
| Absorption | 60 CC of NACL in testing +/− 10 cc |

The following provides specific embodiments of the diaper 100 as constructed for the Very Low Birth Weight Infant. Referring now to FIGS. 2 and 3 certain dimension of the diaper 100 are important to the effectiveness of the developmentally designed diaper.

Diaper Specifications for the LBW Infant

| | |
|---|---|
| Weight | 17 grams +/− 3.5 gm |
| Pad length (L₁) | 25 cm +/− 3.5 cm |
| Pad width (P₁, P₂) | 10 cm +/− 2.0 cm |
| Pad crotch size (P₂ × P₄) | 6 cm × 5 cm +/− 2.5 cm |
| Pad material | chlorine free sulphate cellulose pulp |
| Outer layer material | Non woven material |
| Non woven length (L₂) | 29 cm +/− 2.5 in |
| Non woven width (D₁, D₃) | 19 cm +/− 3.0 cm |
| Non woven crotch width (D₂) | 14.5 cm +/− 3 cm |
| Elastic | Double elastic 29 cm +/− 3.5 cm |
| Inner liner | 13 cm × 8 cm +/− 2.5 cm |
| Absorption | 90 CC of NACL in testing +/− 10 cc |

While the principles of the invention have been described in connection with specific diaper, it is to be understood that this description is not a limitation on the scope of the invention.

1. A developmentally designed diaper for extremely low birth weight infants comprising:
an outer layer having the dimensions L₂, D₁, D₂, and D₃;
an absorbent pad having the dimensions L₁, P₁, P₂, P₃ and P₄;
a wick away liner disposed on top of the absorbent pad; and
an absorbent inner liner that pulls moisture away from an infants skin to which it makes contact past the wick away liner and into the absorbent pad;
wherein:
L₁=15 cm
P₁ and P₃=7 cm
P₂=2.5 cm
P₄=2 cm
L₂=19 cm
D₁ and D₃=15 cm
D₂=10.5 cm.

2. The diaper of claim 1, further comprising elastic members having a dimension of about 10 cm in length extending in the longitudinal direction and interposed and secured between the absorbent inner liner and a back-sheet along respective side edges of the diaper.

3. The diaper of claim 1, wherein the absorbent liner is between about 11.5 cm×6.0 cm in dimension.

4. The diaper of claim 1, wherein absorbent pad has an absorption capability of about 40 CC of NACL in testing.

5. The diaper of claim 1, wherein the absorbent pad comprises chlorine free sulphate cellulose pulp.

6. The diaper of claim 1, wherein the outer layer comprises a non woven material.

7. The diaper of claim 1, wherein the absorbent inner liner comprises a non woven inner liner.

8. A developmentally designed diaper for very low birth weight infants comprising:
an outer layer having the dimensions L₂, D₁, D₂, and D₃;
an absorbent pad having the dimensions L₁, P₁, P₂, P₃ and P₄;
a wick away liner disposed on top of the absorbent pad; and
an absorbent inner liner that pulls moisture away from an infants skin to which it makes contact past the wick away liner and into the absorbent pad;
wherein:
L₁=20.5 cm
P₁ and P₃=9 cm
P₂=4 cm
P₄=3 cm
L₂=24 cm
D₁ and D₃=19 cm
D₂=12 cm

9. The diaper of claim 8, further comprising elastic, members having a dimension of about 16 cm in length extending, in the longitudinal direction and interposed and secured between the absorbent inner liner and a back-sheet along respective side edges of the diaper.

10. The diaper of claim 8, wherein the absorbent liner is between about 13 cm×6 cm in dimension.

11. The diaper of claim 8, wherein the absorbent pad has an absorption capability of about 60 CC of NACL in testing.

12. The diaper of claim 8, wherein the absorbent pad comprises chlorine free sulphate cellulose pulp.

13. The diaper of claim 8, wherein the outer layer comprises a non woven material.

14. The diaper of claim 8, wherein the absorbent inner liner comprises a non woven inner liner.

15. A developmentally designed diaper for low birth weight infants comprising:
an outer layer having the dimensions L₂, D₁, D₂, and D₃;
an absorbent pad having the dimensions L₁, P₁, P₂, P₃ and P₄;
a wick away liner disposed on top of the absorbent pad; and
an absorbent inner liner that pulls moisture away from an infants skin to which it makes contact past the wick away liner and into the absorbent pad;
wherein:
L₁=25 cm
P₁ and P₃=10 cm
P₂=6 cm
P₄=5 cm
L₂=29 cm
D₁ and D₃=19 cm
D₂=14.5.

16. The diaper of claim 15, further comprising elastic members having a dimension of about 29 cm in length extending in the longitudinal direction and interposed and secured between the absorbent inner liner and a back-sheet along respective side edges of the diaper.

17. The diaper of claim 15, wherein the absorbent liner is between about 13 cm×8 cm in dimension.

18. The diaper of claim 15, wherein the absorbent pad has an absorption capability of about 90 CC of NACL in testing.

19. The diaper of claim 15, wherein the absorbent pad comprises chlorine free sulphate cellulose pulp.

20. The diaper of claim 15, wherein the outer layer comprises a non woven material.
